(19) United States
(12) Patent Application Publication
(10) Pub. No.: US 2006/0246423 A1
(43) Pub. Date: Nov. 2, 2006

(54) Method and kit for the collection and maintenance of the detectability of a plurality of microbiological species in a single gynecological sample

(76) Inventors: Martin E. Adelson, Hillsborough, NJ (US); Eli Mordechai, Robbinsville, NJ (US)

Correspondence Address: MEDICAL DIAGNOSTIC LABORATORIES LLC, 2439 KUSER ROAD, HAMILTON, NJ, 08690, US

(21) Appl. No.: 11/343,858

(22) Filed: Jan. 31, 2006

Related U.S. Application Data
(60) Provisional application No. 60/651,688, filed on Feb. 10, 2005.
(60) Provisional application No. 60/654,485, filed on Feb. 18, 2005.
(60) Provisional application No. 60/654,729, filed on Feb. 18, 2005.

(51) Int. Cl. C12Q 1/70; G01N 33/569; G01N 33/554; A61M 35/00; G06F 19/00; C12M 1/34; C12Q 1/04

(52) U.S. Cl. 435/5; 702/19; 435/7.32; 435/7.31; 435/287.2; 604/1; 435/34

(57) ABSTRACT

A method and kit related thereto are described for the collection and maintenance of detectability of a plurality of species of microbiological agents selected from the group consisting of bacteria, fungi, viruses, and protozoa, in a single gynecological sample which comprises providing transport media in a resealable container and instructions for preparation and handling of the gynecological sample and a written indication of the detectability of a plurality of species.

### CROSS REFERENCES TO RELATED APPLICATIONS

The present application claims benefit, under 35 U.S.C. 119(e), to U.S. Provisional Application No. 60/651,688, entitled “A Method and Kit for the Collection and Maintenance of the Detectability of a Plurality of Microbiological Species in a Single Gynecological Sample,” filed on Feb. 10, 2005, the entire contents of which are hereby incorporated by reference. Additionally, the present application claims benefit, under 35 U.S.C. 119(e), to U.S. Provisional Application No. 60/654,485, entitled “Integrated Method for Collection and Maintenance of Detectability of a Plurality of Microbiological Agents in a Single Clinical Sample and for Handling a Plurality of Samples for Reporting a Sum of Diagnostic Results for Each Sample,” filed on Feb. 18, 2005, the entire contents of which are hereby incorporated by reference. Also, the present application claims benefit, under 35 U.S.C. 119(e), to U.S. Provisional Application No. 60/654,729, entitled “A Method of Receiving and Handling a Plurality of Clinical Samples for Reporting a Sum of Diagnostic Results for Each Sample,” filed on Feb. 18, 2005, the entire contents of which are hereby incorporated by reference.

### BACKGROUND OF THE INVENTION

1. Field of the Invention

The present invention is in the field of clinical diagnostic services, i.e., in the industry of identification, characterization, and quantification of biological agents associated with disease conditions. The present invention is particularly directed toward a method and materials for the collection and maintenance of detectability of a plurality of species of microbiological agents selected from the group consisting of bacteria, fungi, viruses, and protozoa in a single clinical sample.

2. Description of the Related Art

Clinical diagnostics provide an essential aid to the physician for the diagnosis and monitoring of numerous pathologies and infectious diseases. Rapid and accurate identification of causative agents of a myriad of different human pathophysiological conditions is a paramount requisite to effective treatment.

A biological sample generally is taken from the patient, most often at the request of a physician, and sent to a medical laboratory for analysis to establish or confirm a diagnosis of clinical symptoms. A physician may suspect a particular causative agent upon physical examination. However, certain symptoms may be characteristic of a plethora of different causative agents. Therefore, due to misdiagnoses of causative agents, patients may be treated non-efficaciously. Moreover, in other instances, a physician may request a certain diagnostic test to be performed on a clinical specimen wherein the test subsequently produces a negative result. Then, of course, further clinical samples and diagnostic testing are required. In many instances, due to the lack of timely and accurate diagnoses, patients' original conditions progress to the further detriment of treatability and to the well-being of the patient. Accurate clinical diagnosis is critical to specifically identify causative agents in a timely manner which mediates pathophysiological conditions. Accordingly, a need indeed exists for materials and a method, for example, to collect an accurate clinical sample, e.g. a “snapshot,” representative of vaginal flora, i.e., a certain gynecological microbiological environment, and maintain the detectability of a plurality of species in a single gynecological sample.

Diagnostic kits are available, for example, capable of detecting several specific species. However, in many instances current diagnostic products and services are inadequate to identify the causative agent or are inoperable under clinical circumstances.

Advances in the detection of C. trachomatis and N. gonorrhoeae, for example, have included the development of nucleic acid amplification tests from cervical as well as urine samples. The Roche COBAS AMPLICOR™ CT/NG Test, for example, is an in vitro multiplex diagnostic test that can detect either or both Chlamydia trachomatis or Neisseria gonorrhoeae in endocervical or urethral swabs and/or urine samples. The COBAS AMPLICOR™ Analyzer is an instrument which automates amplification and detection of the PCR process. The test utilizes polymerase chain reaction (PCR) nucleic acid amplification and nucleic acid hybridization (Roche Diagnostic Systems, Branchburg, N.J.). APTIMA COMBO 2 Assay is a Gen-Probe nucleic acid amplification test that uses target capture for in vitro qualitative detection and differentiation of rRNA from C. trachomatis and N. gonorrhoeae in endocervical and male urethral swab specimens and in urine specimens. The assay uses target capture (TC), Transcription-Mediated Amplification (TMA) and Dual Kinetic Assay (DKA) (Gen-Probe, Inc., San Diego, Calif.).

However, in view of the myriad of different pathological agents that mediate disease conditions, a need indeed exists for materials and methods to collect an accurate clinical sample, e.g. a “snapshot,” representative of a certain gynecological microbiological environment, for example, and maintain the detectability of the diversity of pathological agents in a single gynecological sample.

### BRIEF SUMMARY OF TIE INVENTION

The present invention is directed to methods and materials for the collection and maintenance of detectability of a plurality of species of microbiological agents indicative of a gynecological disorder selected from the group consisting of bacteria, fungi, viruses and protozoa, in a single gynecological sample comprising providing transport media in a resealable container, a sterile swab, and instructions for preparation and handling of a gynecological sample, and an indication of the detectability of the plurality of species.

The current invention is particularly directed to methods and materials wherein the plurality of species includes at least three (3), four (4), five (5), or six (6) species selected from the group consisting of Bacteroides fragilis, Candida albicans, Candida glabrata, Candida parapsilosis, Candida tropicalis, Chlamydia trachomatis, Gardnerella vaginalis, Haemophilis ducreyi, Herpes simplex virus subtype 1 (HSV-1), Herpes simplex virus subtype 2 (HSV-2), Human papillomavirus (HPV), Mobiluncus mulieris, Mobiluncus curtisii, Molluscum contagiosum Virus, Mycoplasma genitalium, Mycoplasma hominis, Neisseria gonorrhoeae, Treponema pallidum, Trichomonas vaginalis, Ureaplasma urealyticum, and Streptococcus agalactiae (Group B Streptococcus).

Certain embodiments of the present invention of this type are wherein the plurality of species comprises Chlamydia trachomatis and Neisseria gonorrhoeae.

Further embodiments of the present invention of this type are wherein the plurality of species comprises Chlamydia trachomatis, Neisseria gonorrhoeae and Trichomonas vaginalis.

Still further embodiments of the present invention of this type are wherein the plurality of species comprises Gardnerella vaginalis, Mobiluncus mulieris, Mobiluncus curtisii, and Bacteroides fragilis.

Embodiments of the present invention are also, for example, wherein the plurality of species comprises Candida albicans, Candida glabrata, Candida parapsilosis, and Candida tropicalis.

Further embodiments of the present invention are wherein the plurality of species comprises Mycoplasma genitalium, Mycoplasma hominis, and Ureaplasma urealyticum.

Embodiments of the present invention are wherein the plurality of species comprises, Herpes simplex virus, Treponema pallidum, and Haemophilis ducreyi.

Particularly preferred embodiments of the present invention include each of the embodiments referred to supra wherein the plurality of species comprises at least one species selected from the group consisting of Molluscum contagiosum Virus, Mycoplasma genitalium, and Mycoplasma hominis.

The invention is further directed to a compilation of materials or an article of manufacture, i.e., a kit for the collection and maintenance of detectability of a plurality of species of microbiological agents selected from the group consisting of bacteria, fungi, viruses, and protozoa, in a single gynecological sample comprising transport media in a resealable container, a sterile swab, and instructions for preparation and handling of a gynecological sample and an indication of the detectability of the plurality of species.

### BRIEF DESCRIPTION OF THE DRAWINGS

FIG. 1 is an example of a Test Requisition Form.

FIG. 2 displays validation data for Bacteroides fragilis wherein the PCR amplicon is 842 bp in which each sample was independently inoculated and extracted in triplicate. Lanes 2-4 represent detectability after storage at room temperature for zero days. Lanes 5-7 represent detectability after storage at room temperature for one day. Lanes 8-10 represent detectability after storage at room temperature for two days. Lanes 11-13 represent detectability after storage at room temperature for three days. Lanes 14-16 represent detectability after storage at room temperature for four days. Lanes 17-19 represent detectability after storage at room temperature for five days. Lanes 20 and 21 are the positive and negative controls, respectively.

FIG. 3 displays validation data for Mobiluncus mulieris wherein the PCR amplicon is 1015 bp in which each sample was independently inoculated and extracted in triplicate. Lanes 2-4 represent detectability after storage at room temperature for zero days. Lanes 5-7 represent detectability after storage at room temperature for one day. Lanes 8-10 represent detectability after storage at room temperature for two days. Lanes 11-13 represent detectability after storage at room temperature for three days. Lanes 14-16 represent detectability after storage at room temperature for four days. Lanes 17-19 represent detectability after storage at room temperature for five days. Lanes 20 and 21 are the positive and negative controls, respectively.

FIG. 4 displays validation data for Candida albicans.

FIG. 5 displays validation data for Candida glabrata.

FIG. 6 displays validation data for Candida parapsilosis.

FIG. 7 displays validation data for Candida tropicalis.

FIG. 8 displays validation data for Chlamydia trachomatis.

FIG. 9 displays validation data for Gardnerella vaginalis.

FIG. 10 displays validation data for Haemophilis ducreyi.

FIG. 11 displays validation data for HSV-1.

FIG. 12 displays validation data for HSV-2.

FIG. 13 displays validation data for Trichomonas vaginalis.

FIG. 14 displays validation data for Ureaplasma urealyticum.

### DETAILED DESCRIPTION

Unless defined otherwise, all technical and scientific terms used herein have the same meaning as is commonly understood by one of skill in the art to which this invention belongs. All publications and patents referred to herein are incorporated by reference.

Physicians are generally faced with observing patients' symptoms, obtaining biological samples, and ordering clinical diagnostic tests to determine the identity of causative agents which mediate pathological conditions. Since methods of treatment of pathophysiological conditions are intimately related to the identity of the causative agent(s) of the condition, rapid and accurate identification and reporting of the causative agent(s) is of paramount importance to the practice of medicine today. The present invention enables the accurate and rapid reporting of the detection of any of a plurality of biological agents from each clinical sample.

It is an object of the present invention to provide methods and materials for the collection and maintenance of detectability of a plurality of species of microbiological agents indicative of a gynecological disorder selected from the group consisting of bacteria, fungi, viruses, and protozoa, in a single gynecological sample comprising providing transport media in a resealable container, a sterile swab, instructions for preparation and handling of a gynecological sample, and an indication of the detectability of the plurality of species.

Proper and timely identification of causative agent(s) of vaginosis, a massive microecologic alteration of the vaginal flora, is a continuous problem for obstetrician-gynecologists. Bacterial vaginosis (BV), for example, is related to considerable and preventable infectious morbidity in non-pregnant women. Bacterial vaginosis mediates endometritis, pelvic inflammatory disease, post-surgical abortion infections, post-hysterectomy infections, an increased risk of HIV acquisition and cervical intraepithelial neoplasia. The diagnosis of BV is complicated by the polymicrobial nature of the condition. Bacterial Vaginosis (BV), for example, is characterized by a logarithmically increased concentration of Gardnerella vaginalis and logarithmically increased concentrations of pathogenic bacteria, including Bacteroides spp., Mobiluncus spp., along with Ureaplasma urealyticum and Mycoplasma spp. Bacteroides fragilis is an anaerobic bacterium that is commonly associated with BV. Mobiluncus species, e.g., Mobiluncus mulieris and Mobiluncus curtisii, are anaerobic bacteria that are commonly associated with BV. Fifty percent of patients diagnosed with BV, however, display no symptoms.

Mycoplasmas are small (0.2-0.3 nm) membrane bound organisms capable of independent self-replication. The most prevalent strains recoverable from the genital tract are Ureaplasma urealyticum, Mycoplasma hominis and Mycoplasma genitalium. In some pregnant women, Ureaplasma infections are considered to be the cause of chorioamnionitis and premature delivery. They are frequently transmitted from mothers to their infants, which may cause a variety of disorders including pneumonia, persistent pulmonary hypertension, and chronic infection of the central nervous system. M. hominis is associated with pyelonephritis, pelvic inflammatory disease (PID), spontaneous abortion, and postpartum septicemia and fever. M. genitalium has been associated with non-gonoccocal urethritis, acute endometritis, cervicitis, and pelvic inflammatory disease (PID). Infants become colonized with genital mycoplasmas during birth. Genital mycoplasma infections are usually diagnosed by culture. However, due to its fastidious slow-growing nature, M. genitalium, for example, may take up to eight (8) weeks to culture.

Candida Vaginitis (CV) is currently the second most common cause of vaginal infections, with bacterial vaginosis the most common diagnostic entity. However, CV is misdiagnosed in as much as 50% of all cases. Most studies indicate that CV is a frequent diagnosis among young women, affecting as many as 15% to 30% of symptomatic women visiting a clinician. Candida albicans is one of the major causes of Candida Vaginitis (CV). The widespread use of topical antifungals appears to contribute to selection for non-albicans yeasts, e.g., C. glabrata accounts for 7% of all vaginal fungal infections and about 10% of vaginal yeast isolates. Candida tropicalis is isolated from 1% to 5% of vaginal yeast isolates and may be associated with a higher rate of recurrence after standard treatment. C. parapsilosis accounts for 1% of vaginal yeast isolates.

Chlamydia trachomatis is the causative agent of a variety of diseases including trachoma and urogenital infections. It is the most common sexually transmitted bacterial agent and in women it causes cervicitis, urethritis, endometritis and salpingitis. In more complicated cases it may result in tubal scarring, infertility, and ectopic pregnancy. In men it causes urethritis and proctatitis. Other forms of infection also seen are trachoma, the most preventable form of blindness, and conjunctivitis in neonates.

Neisseria gonorrhoeae is the causative agent of the sexually transmitted disease gonorrhea. It is the most frequently reported communicable disease in the United States. In women, the most common presentation is endocervical infection. If left untreated it may develop into vulvovaginitis, salpingitis, and pelvic inflammatory disease (PID). Infections in men range from uncomplicated lower genital tract involvement such as urethritis, to the more serious epididymitis, prostatitis, and urethral stricture. Untreated asymptomatic infections may, in certain instances, develop into disseminated gonococcal infection (DGI).

Genitourinary tract infections due to C. trachomatis and N. gonorrhoeae are a major cause of morbidity in sexually active individuals. In males they may cause epididymitis and urethritis. In females, they can cause pelvic inflammatory disease (PID), ectopic pregnancy, and infertility. If left untreated, N. gonorrhoeae may develop into a disseminated gonococcal infection (DGI). Coinfection with C. trachomatis and N. gonorrhoeae is not uncommon. In fact, up to half of patients diagnosed with infection of one of these pathogens may be infected with the other and, therefore, it is important to test all sexually active individuals for both. Both sexually transmissible pathogens are detectable by swabbing the urethra and/or the cervix (for women) and performing either a culture and/or a nucleic acid amplification assay (see Van Doornum et al., 2001, Journal of Clinical Microbiology 39(3):829-835).

Chlamydia trachomatis, Neisseria gonorrhoeae, and Trichomonas vaginalis are the major causes of leukorrhea. Neisseria gonorrhoeae is the causative agent of the sexually transmitted disease gonorrhea. In women, the most common symptom of N. gonorrhoeae infection is endocervical infection, and if left untreated, may develop into vulvovaginitis and pelvic inflammatory disease. As a protozoan parasite, Trichomonas vaginalis is the causative agent of the sexually transmitted disease trichomoniasis. T. vaginalis infection is the primary cause of vaginitis, cervicitis and urethritis in women. Routine clinical diagnosis usually depends on microscopic identification of the parasite in wet mount preparations, which are only 60% sensitive as compared to culture-positive women.

The three major causes of Genital Ulcer Disease (GUD) are Herpes simplex virus, Treponema pallidum (syphilis), and Haemophilis ducreyi (chancroid). As treatment options vary, it is medically necessary to identify the causative agent of GUD. Currently, the diagnosis of GUD is based primarily on the clinical presentation of the ulcer itself. However, agent-specific diagnosis based solely on the clinical evaluations are often obscured by multiple and mixed infections. T. pallidum is the causative agent of the sexually transmitted disease syphilis. T. pallidum is one of the few major bacterial pathogens of humans that cannot be cultivated on artificial medium.

Human Papillomavirus (HPV) subtyping is of clinical significance in view of the growing evidence for the association of Human Papillomavirus (HPV) subtypes (i.e. HPV-16 and HPV-18) with cervical and ovarian cancer. Particularly, 90% of individuals with major grade cervical intraepithelial neoplasia (CIN 2 and CIN 3) and invasive carcinoma of the cervix are also infected with HPV-16 or HPV-18. Moreover, as much as 10% to 20% of women in the United States have tested positive to HPV by Papanicolaou (Pap) smear. Since Pap smears cannot differentiate among HPV subtypes, asymptomatic individuals that are HPV positive are being disregarded by the clinicians.

Molluscum contagiosum virus (MCV) is a member of the human pox viruses which produces small raised papules or lesions with central umbilications and a white, firm, curd-like core. Infection occurs during sexual intercourse. MCV is a common infection in the United States and accounts for approximately 1% of all undiagnosed skin disorders. Many physicians find it necessary to differentiate MCV from Human papillomavirus (HPV) or Herpes simplex virus (HSV) infections which have greater mortality and morbidity.

Vaginal Group B Streptococcus (Streptococcus agalactiae) (GBS), the most common cause of life-threatening infection in newborns, is a common cause of neonatal septicemia, pneumonia and meningitis. One out of every twenty babies with GBS dies from the infection. In pregnant women, GBS can cause bladder infections, womb infections, and stillbirth. Many adults are asymptomatic carriers of GBS in the bowel, vagina, bladder or throat. Diagnosis by traditional cultures may take several days to complete. However, once properly diagnosed, GBS can be treated with antibiotics to prevent the spread from mother to baby.

### Exemplary Embodiment

The Applicants describe herein methods and materials to collect and maintain the detectability of multiple pathogens from a single gynecological swab. Specimen viability has been validated for up to five days, for example. Longer viability, however, e.g., about 5-10 days, is reasonably expected in view of the data presented herein. No refrigeration is required. The present invention is not drawn to methods for the detection, identification or diagnosis, per se, of any particular microbiological species, or series of species, or disease condition. In contrast, methods and articles of manufacture, i.e., kits, of the present invention are particularly provided for the collection and maintenance of detectability of a plurality of different microbiological species associated with pathological gynecological conditions so that a clinical diagnostic service provider, for example, may enable a physician to collect a single swab specimen (clinical sample) and order any of a plurality of tests on that sample from the clinical diagnostic service provider, for example. The tests, per se, however, whatever tests are used, are not relevant to the subject matter of the present invention. The Applicants indeed present validation data herewith merely to demonstrate the viability of the species exemplified herein using the materials and methods of the present invention. Since the species exemplified herein are well characterized in the art and are, as discovered and illustrated by the Applicants, viable under the conditions described herein they are indeed detectable by an array of different methods, e.g., nucleic acid amplification, known to those of skill in the art.

A method is particularly preferred for the collection and maintenance of detectability of a plurality of species of microbiological agents selected from the group consisting of bacteria, fungi, and viruses, in a single gynecological sample comprising providing transport media in a resealable container, a sterile swab, and instructions for preparation and handling of a gynecological sample and an indication of the detectability of the plurality of species.

“Species of microbiological agents” as used herein refer to microbiological agents that are causative or are otherwise associated with gynecological disorders. Since so many different species of microbiological agents mediate, or are associated with, or are indicative of gynecological disorders, the present invention provides a means for sampling a gynecological environment and preserving an accurate representation of the ambient population of microbiological agents at the time the sample is taken. Particularly, the method of the present invention provides materials for the collection and maintenance of a “snapshot” of a gynecological environment for the detectability of a plurality of species of microbiological agents in a single gynecological sample.

Methods described herein comprise providing a transport media in a resealable container, a sterile swab, and instructions for preparation and handling of a gynecological sample and an indication of the detectability of the plurality of species.

Methods described herein comprise (1) providing a transport media in a resealable container, a sterile swab, and instructions for preparation and handling of a gynecological sample and a written indication of the detectability of the plurality of species, e.g., a test requisition form, and (2) receiving the completed gynecological sample in a package with a completed test requisition form.

Transport media for use in methods of the present invention is a universal transport media in which viability of a plurality of organisms, e.g., bacteria, fungi, and viruses, can be sustained under normal conditions without refrigeration for at least 48 hours. Transport media for use in methods of the present invention is preferred wherein the detectability of species selected from the group consisting of Bacteroides fragilis, Candida albicans, Candida glabrata, Candida parapsilosis, Candida tropicalis, Chlamydia trachomatis, Gardnerella vaginalis, Haemophilis ducreyi, Herpes simplex virus subtype 1 (HSV-1), Herpes simplex virus subtype 2 (HSV-2), Human papillomavirus (HPV), Mobiluncus mulieris, Mobiluncus curtisii, Molluscum contagiosum Virus, Mycoplasma genitalium, Mycoplasma hominis, Neisseria gonorrhoeae, Treponema pallidum, Trichomonas vaginalis, Ureaplasma urealyticum, and Streptococcus agalactiae (Group B Streptococcus) is maintained at room temperature for five (5) days.

An example of a general support media for a variety of organisms in a clinical sample is Stuart's medium (see, e.g., Stuart et al., 1954, The problem of transport of specimens for culture of Gonococci. Canadian Journal of Public Health. 45(2):73-83). Stuart's medium is a well-known buffered transport medium which includes the component sodium glycerophosphate to permit minimal multiplication and sodium thioglycollate as a reducing agent to prevent oxidation within the sample. Stuart's medium, however, contains no nutrients. The absence of nutrients retards the growth of commensal organisms within the sample which can multiply and overgrow the less hardy pathogens. However, the absence of nutrients in Stuart's media can be detrimental to the viability of less hardy pathogens. Preferred transport medium comprises an aqueous balanced salt solution buffered at approximately physiological pH, at least one protein stabilizer, and combinations of carbohydrate and amino acid nutrient sources. The medium is buffered to maintain physiological pH and may include a pH indicator in order to indicate variation of pH outside the physiological pH range. The medium further comprises antimicrobial and antifungal agents and can include gelatin. Transport media, for example, is liquid media wherein detectability of a plurality of species of microbiological agents including bacteria, fungi, and viruses is maintainable under normal conditions, without refrigeration, for at least 72 hours. Example transport media herein is demonstrated to be suitable for maintaining the detectability, for example, of a plurality of species of microbiological agents including bacteria, fungi, and viruses under normal conditions, without refrigeration, for about five (5) days (e.g., UTM-RT Transport medium, Copan Diagnostics Inc., Corona, Calif.).

Transport medium, for example, consists of modified Hank's balanced salt solution supplemented with bovine serum albumin, cysteine, gelatin, sucrose, and glutamic acid. The pH is buffered with HEPES buffer, for example. Phenol red is used to indicate pH. Vancomycin, amphotericin B, and colistin are incorporated in the medium to inhibit growth of competing bacteria and yeast. The medium is isotonic and non-toxic to human cells. Example components comprise, for example, Hank's Balanced Salts, Bovine Serum Albumin, L-Cysteine, Gelatin, Sucrose, L-Glutamic Acid, HEPES Buffer, Vancomycin, Amphotericin B, Colistin, Phenol Red; pH 7.3±0.2 @ 25° C. Antimicrobial compositions can include vancomycin, gentamicin, colistin, or amphotericin B.

A general transport medium of about 1.0 liter (total volume) may be prepared in the following aqueous composition: gelatin, 0-20.0 g; sugar, 65-75 g; HEPES, 5-7 g; KCl, 0.3-0.6 g; L-glutamic acid, 0.5-1.0 g; phenol red, 5-15 mg; CaCl₂, 0.1-0.5 g; MgSO₄.7H₂O, 0.1-0.3 g; bovine serum albumin V, 1.0-20.0 g; vancomycin, 0.01-0.05 g; colistin, 100,000-250,000 units; and amphotericin B, 0.5-3.0 mg.

An example may include the following ingredients in about 995 ml of water q.s. 1 liter: sucrose, 68.46 g; HEPES, 5.96 g; KCl, 0.4 g; L-glutamic acid, 0.72 g; phenol red, 11.0 mg; CaCl₂, 0.27 g; MgSO₄.7H₂O, 0.20 g; BSA, 5.0 g; gelatin, 5.0 g; vancomycin, 0.025 g; colistin, 200,000 units; and amphotericin B, 1.0 mg. The pH of the medium can be adjusted with acidic or basic solutions to arrive at a final pH within physiological limits (see, e.g., U.S. Pat. No. 5,702,944, entitled Microbial Transport Media, which is herein incorporated by reference in its entirety).

Preferred commercially available and validated examples of transport medium for use in methods and articles of the present invention include, for example, the following: UTM-RT Transport Medium, BD Cellmatics Viral Transport Medium® (Becton, Dickinson & Company, Sparks, Md.), Multitrans Culture Collection and Transport System (Starplex Scientific, Etobicoke, Ontario, Canada), The ThinPrep® Pap Test Preservcyt® Solution (CYTYC Corporation, Boxborough, Mass.), SurePath® (Tripath Imaging Inc., Burlington, N.C.).

Preferred methods of the present invention provide a transport media in a resealable container, a sterile swab, instructions for preparation and handling of a gynecological sample and a written indication of the detectability of the plurality of species.

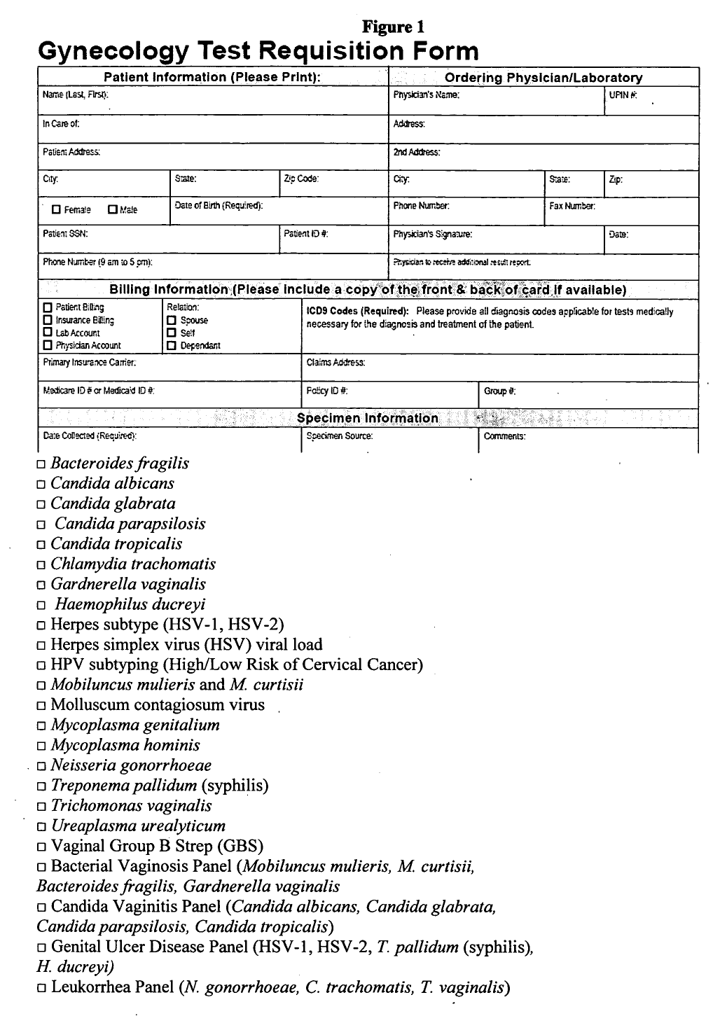

A labeled screw-cap tube is preferred, for example, as a resealable container, which contains a volume of transport medium, between about 1 ml and about 5 ml, for example, suitable for accurate collection and maintenance of a population of microbiological agents representative of a gynecological environment. A resealable container optionally contains glass beads (three 3 mm beads, for example). Although not required to be provided, per se, in methods and the specified compilation of materials described herein, at least one sterile swab, well-known in the art of gynecology, is preferred in the materials of a packaged kit described herein for obtaining the gynecological sample. At least one sterile swab for obtaining the gynecological sample, for example, is preferably supplied with the transport media in a package along with written instructions for preparation and handling of a gynecological sample in the media, and a written indication of the detectability of a plurality of species, e.g., a test requisition form. The transport medium may be supplied, however, alone in a package which comprises a writing, i.e., a written indication of the detectability of a plurality of species, for example, a test requisition form (see FIG. 1) which lists a plurality of species of microbiological agents described herein for selection, for example, by the attending physician. The compilation of materials described herein is preferably provided in methods described herein in a container, i.e., a package. The package preferably contains the materials in a kit-format. Written instructions for preparation and handling of a gynecological sample in the media are preferred to be included in the compilation of materials otherwise described herein as a packaged kit intended, designated, and prepared for the specific purpose of collection and maintenance of detectability of a plurality of species of microbiological agents described herein. Once a swab sample is collected, it should be placed immediately into the transport tube where it comes into contact with transport medium.

A first set of example instructions are as follows:
1. Collect the single specimen with a swab (Polyester (Dacron) tipped swabs are suitable).
2. Aseptically remove the cap from the transport media.
3. Insert swab into the tube with the transport medium.
4. Break swab shaft by bending it against the tube wall.
5. Replace cap to tube and close tightly.
6. Label the tube with appropriate patient information.
7. Complete the test requisition form included herewith.
8. Send these items in the pre-addressed postage materials included herewith to the laboratory for immediate analysis.

A second set of example instructions are as follows:
1. Visualize cervix with speculum without lubricant.
2. Remove mucus and/or secretions from the cervix with a swab, discard swab.
3. Firmly, yet gently, sample the endocervical canal with sterile swab for 10 seconds.
4. Place the swab into the transport vial.
5. Be sure the cap is sealed tightly.

Specimen collection swab options include, for example: one regular size plastic shaft swab with polyester fiber tip; two regular size plastic shaft swabs with polyester fiber tips; one regular size plastic shaft swab and one Minitip plastic shaft swab pre-scored for easy breakage, both with polyester fiber tips; one Minitip plastic shaft swab with polyester fiber tip pre-scored for easy breakage; one Combo stainless steel wire-plastic shaft Minitip swab with polyester fiber tip; one regular size plastic shaft swab and one Combo stainless steel wire-plastic shaft Minitip swab, both with polyester fiber tips. These different swab applicator shafts facilitate the collection of specimens from various sites on a patient.

To Maximize the Success of Clinical Diagnostics Subsequent to Methods Described Herein

A clinical diagnostic laboratory should be physically set up so that specimen separation and extractions occur in a separate room, using a “Class II Biohazard Safety Hood.” PCR preparation should occur in a separate room, within one of many PCR cabinets which are dedicated solely to PCR preparation. The PCR amplification should occur in thermocyclers located in an enclosed room. For post-amplification process of convention PCR reactions, gel electrophoresis should be performed in yet another physically separate room. UV lights should be used in the PCR hoods and commercial solvents, such as DNAway (Molecular Bio Products, San Diego, Calif.), to decontaminate all work surfaces prior to and at the completion of any procedures occurring in that area. Sterile, disposable plasticware should be used wherever possible. All glassware should be autoclaved. All PCR reactions should be performed in individual closed tube systems as opposed to 96-well microtiter plates to eliminate cross contamination. Real-time PCR assays, for example, do not require gel electrophoresis and therefore eliminate post-amplification specimen handling. All technicians should only manipulate one specimen at a time. This means when a reagent is added to a batch of specimens, it occurs one tube at a time. The next patient's reaction tube is not opened until the previous patient's tube has been closed. Pipette tips used when dispensing reagents should be filtered to prevent aerosol contamination and are also replaced between all specimens. Reagents used during PCR preparation may be aliquoted into 1.5 ml microcentrifuge tubes, for example, as opposed to dispensation into stock bottles of greater volumes. This enables the laboratory to monitor potential contamination closely and discard any reagents, if ever necessary. The use of separate rooms is recommended to decontaminate an entire room if contamination is suspected. Positive and negative controls should be employed to assess false positives as well as false negatives. Uracil-N-glycosylase is recommended in every reaction to minimize, if not eliminate, any possible carry-over contamination.

### DNA Extraction

Established procedures for DNA extraction are used (see Example II). In brief, swabs are thoroughly mixed in the transport media contained within the transport vials. 470 μl of transport media is mixed with 25 μl of 10% sodium dodecyl sulfate, and 12 μl of freshly prepared DNase-free proteinase-K (10 mg/ml), then incubated for 2 hours at 55° C. DNA is phenol:chloroform:isoamyl alcohol extracted and recovered by ethanol precipitation. DNA is pelleted, dried in a speed vacuum, and resuspended in 20 μl TE buffer. DNA concentration is calculated by absorbance 260/280 readings and was adjusted to 0.2 μg/μl prior to PCR analysis.

### Primers

Any pair of PCR primers may be employed in methods of the present invention that function to amplify target nucleic acids. The art of selection and synthesis of PCR primers in order to amplify a particular target sequence is indeed well-known to those of ordinary skill in the art. Typically, oligonucleotide primers are about 8 to about 50 nucleotides in length. Primers 12 to 24 nucleotides in length are preferred. Primer pairs that amplify particular nucleic acid molecules can be designed using, for example, a computer program such as OLIGO (Molecular Biology Insights, Inc., Cascade, Colo.). A biotin moiety, for example, is preferably attached to the 5′ end of one of the primers to facilitate sample preparation for “pyrosequencing,” a term which denotes the nucleotide sequencing method described in U.S. Pat. Nos. 6,210,891 and 6,258,568; Ronaghi et al., 1998, A sequencing method based on real-time pyrophosphate. Science 281:363-365; and Ronaghi, 2001, Pyrosequencing sheds light on DNA sequencing. Genome Research 11:3-11. Other entities, however, well known to those of skill in the art, may similarly be incorporated, integrated, or attached to one of the primers to facilitate the isolation of the resulting amplicon for pyrosequencing.

### Real-Time PCR

Quantitative real-time PCR is a preferred method of amplification of a target nucleic acid. Products used to accomplish the methods are commercially available from several manufacturers including, but not limited, to Corbett Research (Mortlake, AU), Cepheid (Sunnyvale, Calif.), BioRad (Hercules, Calif.), and Applied Biosystems (Foster City, Calif.). The Corbett Research Rotor-Gene™ 3000, for example, is a centrifugal, real-time DNA amplification system.

Validation studies exemplified herein are merely a general demonstration of the utility and value of the present invention, namely a method for the collection and maintenance of detectability of a plurality of species of microbiological agents in a single gynecological sample, in the grand scheme of clinical diagnostics. The legitimacy of the PCR method is not a relevant factor, as its utility as an invaluable molecular biological tool has already been well established in the international scientific literature through the publication of thousands of peer-reviewed articles. Particularly, molecular amplification of nucleic acids by means of PCR is well-known to those of ordinary skill in the art, i.e., the ability of the PCR method to detect genetic sequences specific to a target pathogen within a given clinical specimen. The Applicants particularly highlight, however, that the methods described herein, which comprise providing transport media in a resealable container to a physician, clinical lab, or medical institution, with instructions for preparing and handling a gynecological sample, along with a test requisition form which indicates the detectability of a plurality of species described herein, affect the ability of a physician, for example, to collect a single swab sample of a gynecological environment for the maintenance of detectability of a plurality of species of microbiological agents. Example assays designed to test sensitivity, specificity, interference and optimization were performed to validate the operability of the methods and materials described herein, as claimed. In other words, PCR methods or reagents employed to detect microbiological agents are not relevant to the scope of the subject matter of the claims appended hereto. In contrast, the present invention is solely drawn toward methods and certain materials for collection and maintenance of detectability of a plurality of species of microbiological agents in a single gynecological swab sample.

Sensitivity refers to a method's ability to detect very minute amounts of a substance or organism. The frequency of a positive test result in patients who have the disease the test is designed to detect, is expressed mathematically as follows:

$$Sensitivity = \frac{True Positives}{True Positives + False Negatives} \times 100$$

Sensitivity studies were initially performed by purchasing well-characterized, validated organisms from the American Tissue Culture Collection (ATCC, Manassas, Va.). The DNA of the virus, bacteria, or fungi is then extracted and quantitated. Standards of known concentrations are used to determine the assay's ability to detect varying concentrations of genetic material. The extracted DNA is serially diluted to concentrations of 1:10, 1:100, 1:1,000 and 1:10,000. By evaluating the presence of bands in these dilutions of known concentrations, the sensitivity of a particular test can be established. For real-time PCR assays, the fluorescence acquisition profile generated from the amplification of the serial dilutions is analyzed. A region encompassing the genetic target of the assay is generally subcloned into a vector system. Through quantitation of the vector and the optimization of the assay as described infra, as few as 10 genomic equivalents of the pathogen can be reproducibly detected.

Specificity studies were used to assess the quality of the primer selection for the assay by determining if their organisms' DNA will cross-react in any way leading to false positives. Initially as a theoretical test, primers are cross-referenced against the billions of other genetic sequences which have been deposited in the public databases by international researchers and any potential conflicts are avoided. Next as an experimental confirmation, the primers and probes are assayed for their inability to amplify dozens of other known bacterial, viral, and fungal organisms which have been identified as human pathogens. An aliquot of the characterized positive control is also spiked in a suspension consisting of the DNA of numerous other organisms to ensure that the particular pathogen target genome is in no way masked or inhibited by other genomic sequences.

Interference studies are used to determine if other substances inherent to the specimen type will interfere with detection by PCR. Certain effects, such as masking the organism's target to produce a false negative, or cross-reactivity to produce a false positive are analyzed. Characteristics, such as the microcosm of normal flora of the genital tract, the abundance of various proteins found in blood, and natural inhibitors commonly found in other body fluids, such as urine, can all have detrimental effects on the PCR process, unless accounted for during the initial processing and extraction procedures.

Optimization studies are the final step of the validation process. In these assays, the concentrations of various reagents are varied such as template DNA, MgCl₂, and primers, and probes as well as the temperature and duration of each step of the thermocycling parameters to improve the clarity of bands or the intensity of signals, as well as eliminate streaks, multiple banding, or haziness, which can impede the visualization of the PCR products or interpretation of real-time PCR results.

### ADDITIONAL REFERENCES

1. Adelson et al., 2005, Simultaneous detection of herpes simplex virus types 1 and 2 by real-time PCR and pyrosequencing. Journal of Clinical Virology 33:25-34. (manuscript published online on Nov. 14, 2004).

2. Trama et al., 2005, Detection of Candida species in vaginal samples in a clinical laboratory setting. Infectious Diseases in Obstetrics and Gynecology 13(2):63-67.

3. Trama et al., 2005, Detection and identification of Candida species associated with Candida vaginitis by real-time PCR and pyrosequencing. Molecular and Cellular Probes 19(2): 145-152.

4. Trama et al. Analyzing Candida albicans gene mutations that contribute to azole resistance by pyrosequencing. American College of Obstetricians and Gynecologists 52^{nd} Annual Clinical Meeting, May 1-5, 2004, Philadelphia, Pa.

5. Trama et al. Novel technique for identification of vulvovaginal candidiasis by real-time PCR and pyrosequencing. American College of Obstetricians and Gynecologists 52^{nd} Annual Clinical Meeting, May 1-5, 2004, Philadelphia, Pa.

6. Adelson et al., Diagnosis of Neisseria gonorrhea, Chlamydia trachomatis, and Trichomonas vaginalis by real-time PCR. American College of Obstetricians and Gynecologists 52^{nd} Annual Clinical Meeting, May 1-5, 2004, Philadelphia, Pa.

7. Mordechai et al., Prevalency of Candida species associated with Candida vaginitis in the United States. American Society of Microbiology 104^{th} General Meeting, May 23-27, 2004, New Orleans, La., Poster C-108.

8. Adelson et al., Development of a real-time PCR assay for the simultaneous detection of herpes simplex virus types 1 and 2 with confirmation by pyrosequencing technology. American Society of Microbiology 104^{th} General Meeting, May 23-27, 2004, New Orleans, La., Poster C-273.

9. Naurath et al., Detection and quantification of Gardnerella vaginalis by real-time PCR. American College of Obstetricians and Gynecologists 53^{rd} Annual Clinical Meeting. May 7-11, 2005, San Francisco, Calif.

10. Trama et al, Detection of molluscum contagiosum virus by real-time PCR and pyrosequencing. American Society of Microbiology 105^{th} General Meeting, Jun. 5-9, 2005, Atlanta, Ga.

11. Feola et al., Detection of Ureaplasma urealyticum, Mycoplasma hominis, and Mycoplasma genitalium by real-time PCR and pyrosequencing. American Society of Microbiology 105^{th} General Meeting, Jun. 5-9, 2005, Atlanta, Ga.

12. Gygax et al., Erythromycin and clindamycin resistance in Group B Streptococcal clinical isolates. Presented by Dr. Martin E. Adelson at the 45^{th} ICAAC (Interscience Conference on Antimicrobial Agents and Chemotherapy) Meeting in Washington D.C. on Dec. 16, 2005.

13. Adelson et al., Evaluation of UTM-RT for the molecular detection of a plurality of OB/GYN related pathogens. Presented by Dr. Martin E. Adelson at the 45^{th} ICAAC (Interscience Conference on Antimicrobial Agents and Chemotherapy) Meeting in Washington D.C. on Dec. 17, 2005.

### EXAMPLES

### Example I

### Validation Studies

To determine if Copan UTM-RT media is suitable for the molecular amplifications diagnostic testing, the following pathogens were purchased from ATCC and detection assays were performed:

TABLE 1

| | Pathogen | ATCC Catalogue Number |
|---|---|---|
| 1 | Bacteroides fragilis | 23745 |
| 2 | Candida albicans | 18804 |
| 3 | Candida glabrata | 2001 |
| 4 | Candida parapsilosis | 10233 |
| 5 | Candida tropicalis | 13803 |
| 6 | Chlamydia trachomatis | VR-901B |
| 7 | Gardnerella vaginalis | 14018 |
| 8 | Haemophilis ducreyi | 27721 |
| 9 | Herpes Simples Virus - 1 | VR-1544 |
| 10 | Herpes Simples Virus - 2 | VR-734 |
| 11 | Mobiluncus mulieris | 35243 |
| 12 | Mycoplasma hominis | 14027 |
| 13 | Neisseria gonorrhoeae | 27628 |
| 14 | Trichomonas vaginalis | 30246 |
| 15 | Ureaplasma urealyticum | 27618 |

### Simulation of a Positive Clinical Specimen

Pathogens were purchased from ATCC in a lyophilized pellet form. Each pellet was dissolved in five ml of TE-buffer (10 mM Tris, pH 7.5, and 1 mM EDTA) in case of bacteria or yeast liquid media (10 g of yeast extract, 20 g of peptone dissolved in 1 L of distilled water, pH7) in case of fungi. Virus cultures were purchased from ATCC as two ml liquid cultures. Dilutions were subsequently prepared as follows:

TABLE 2

| | Concentration (Designation) | | |
|---|---|---|---|
| | 1:1 (A) | 1:10 (B) | 1:100 (C) |
| Original Resuspension | 600 μl | 60 μl | 6 μl |
| TE Buffer (Bacteria, Virus) or | 0 μl | 540 μl | 594 μl |
| Yeast Liquid Media (Fungi) | | | |

DNA was extracted from 500 μl of A, B, and C dilutions using standard laboratory phenol/chloroform/ethanol precipitation protocols. For positive controls, pathogen-positive clinical specimens were identified from the initial laboratory diagnostic tests and 500 μl of the corresponding original cervical swab media specimen was extracted. Previously validated real-time PCRs for each set of pathogens was performed on DNA extracted from Dilutions A, B, and C as well as the clinical samples. Rotor-Gene software calculated C_{T} values for the three ATCC dilutions and the clinical specimens (Rotor-Gene 3000 instrument). The C_{T} values of the dilutions were compared with that obtained for the clinical specimens and a “simulated dilution” was extrapolated for the subsequent studies of the Copan UTM-RT transport medium. Based upon these studies, the following was selected:

TABLE 3

| | | | TE Buffer | |
|---|---|---|---|---|
| | | ATCC | (Bacteria, | |
| | | Resuspension | Virus) or Yeast | Overall |
| | ATCC | used in this | Liquid Media | Dilution |
| | Resuspension | experiment | (Fungi) | of pellet |
| Bacteria | 5 ml TE buffer | 5 μl | 245 μl | 1:250 |
| | added to pellet | | | |
| Fungi | 5 ml TE buffer | 5 μl | 245 μl | 1:250 |
| | added to pellet | | | |
| Viruses | 2 ml culture from | 2 μl | 198 μl | 1:100 |
| | ATCC | | | |

The Applicants' studies suggest that spiking an ATCC culture (pellet suspended in 5 ml of medium or buffer) diluted at 1:50 simulates the concentration of bacterial and fungal pathogens (i.e., 250-fold dilution of ATCC culture) and 1:100 dilution simulates the viral pathogen (100-fold dilution of ATCC culture) in the clinical sample.

### Studying the Stability of the Pathogen

For validation studies, Copan UTM-RT transport medium (Lot # A 303CS02) as provided by the manufacturer was pooled in a sterile bottle. Based upon the simulated dilutions described above for each pathogen, the following cocktails were prepared:

TABLE 4

Per vial (A, B, & C)

| | | | μl Copan |
|---|---|---|---|
| Cocktail | Pathogens | μl Pathogen* | UTM-RT |
| 1 | Candida albicans | 80 μl | 3840 μl |
| | Neisseria gonorrhoeae | 80 μl | |
| 2 | Candida parapsilosis | 80 μl | 3800 μl |
| | Chlamydia trachomatis | 80 μl | |
| | Herpes Simplex Virus - 1 | 40 μl | |
| 3 | Candida glabrata | 80 μl | 3800 μl |
| | Herpes Simplex Virus - 2 | 40 μl | |
| | Trichomonas vaginalis | 80 μl | |
| 4 | Candida tropicalis | 80 μl | 3760 μl |
| | Mobiluncus mulieris | 80 μl | |
| | Ureaplasma urealyticum | 80 μl | |
| 5 | Bacteriodes fragilis | 80 μl | 3840 μl |
| | Mycoplasma hominis | 80 μl | |
| 6 | Gardnerella vaginalis | 80 μl | 3840 μl |
| | Haemophilis ducreyi | 80 μl | |

*Dilution prepared for each pathogen as detailed in Table 4.

Each cocktail was prepared in triplicate (15 ml tubes) and designated A, B, or C. Pathogen culture solution was added to obtain the desired concentration which mimics the pathogen load in a positive clinical sample (1:250-fold dilution for ATCC bacterial and fungal culture and 1:100-fold dilution for ATCC virus culture). 500 μl of the above mix was transferred to three separate microcentrifuge tubes labeled Day 0 to 5.

Inoculated media vials of each cocktail were incubated at room temperature. At 24 hour intervals starting with Day 0 through Day 5, three microcentrifuge tubes were transferred to −20° C. storage. Aliquots from each vial were extracted for DNA by standard laboratory procedures after Day 5. Conventional and real-time PCR reactions for each pathogen on the appropriate cocktail followed. The summary of results is as follows:

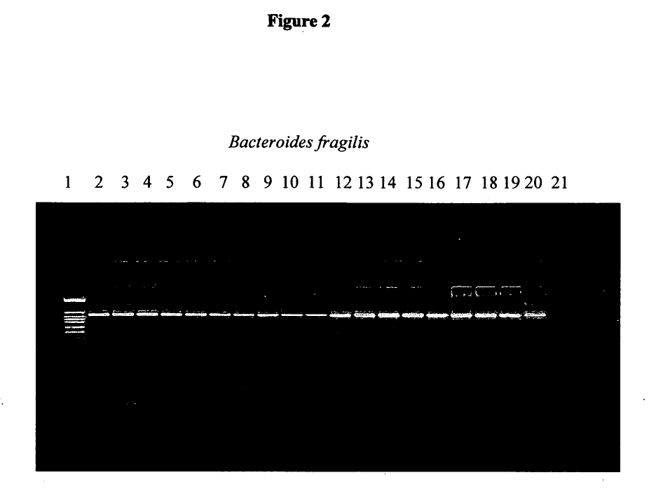

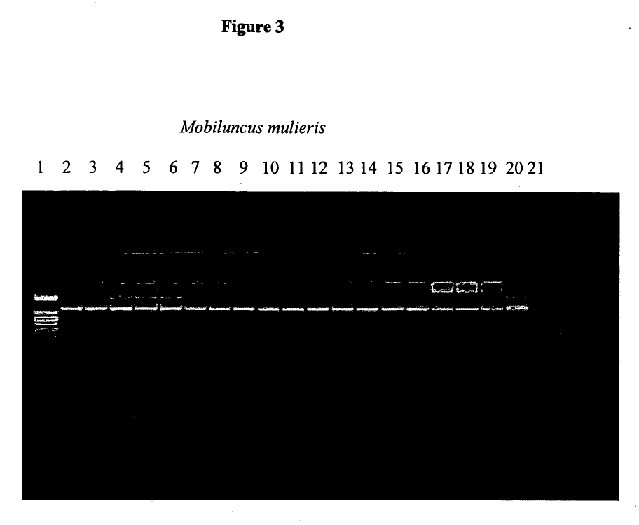

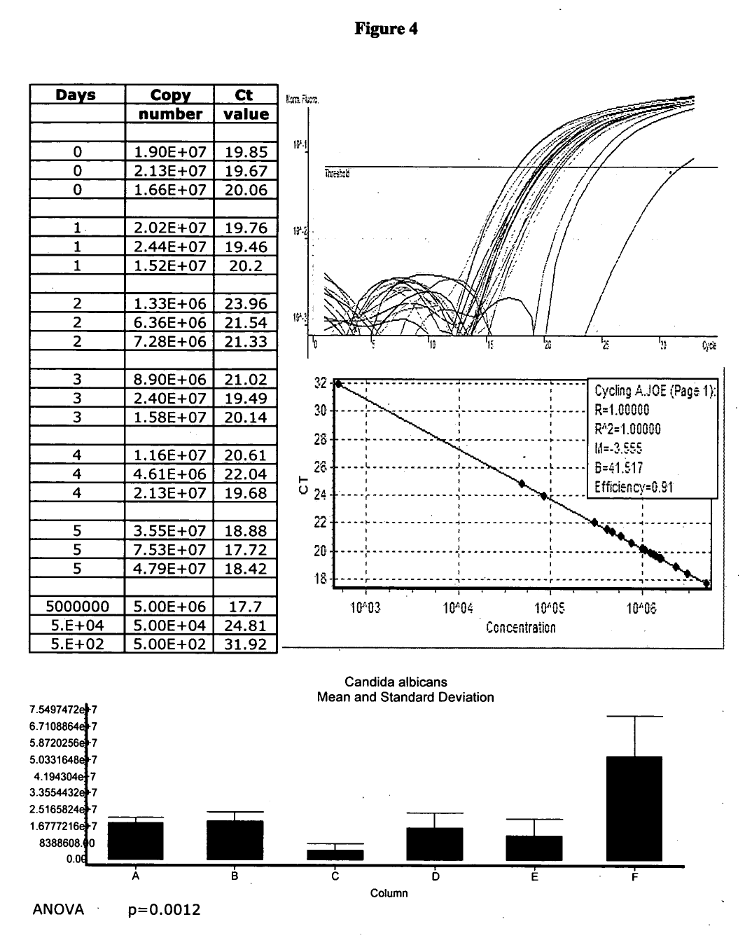

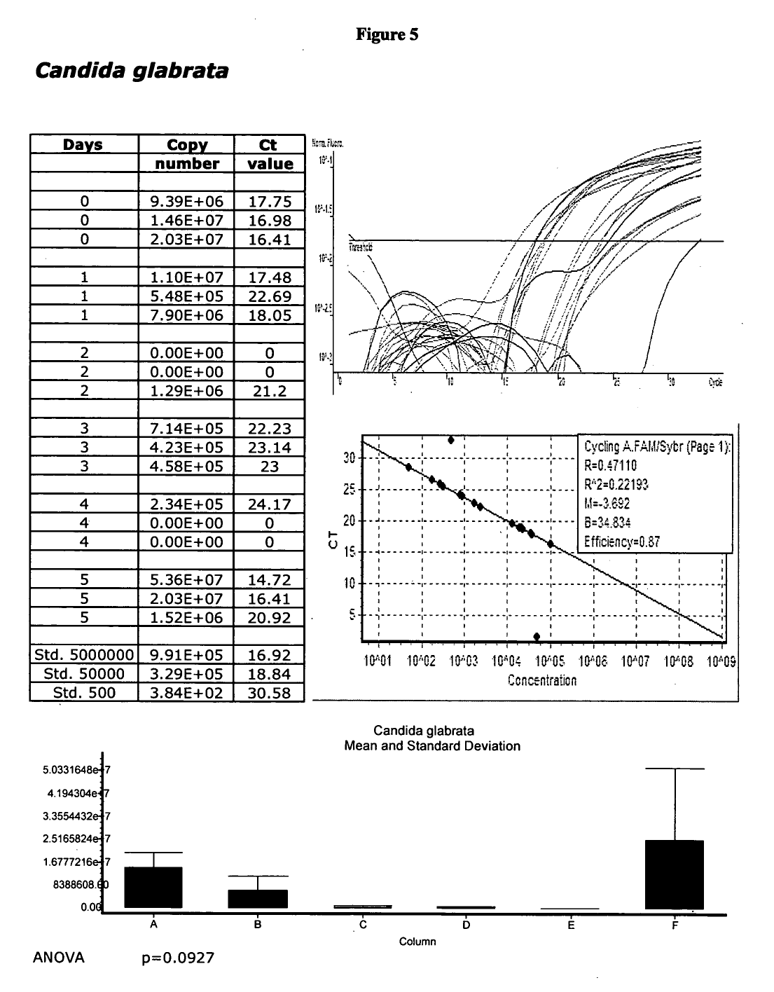

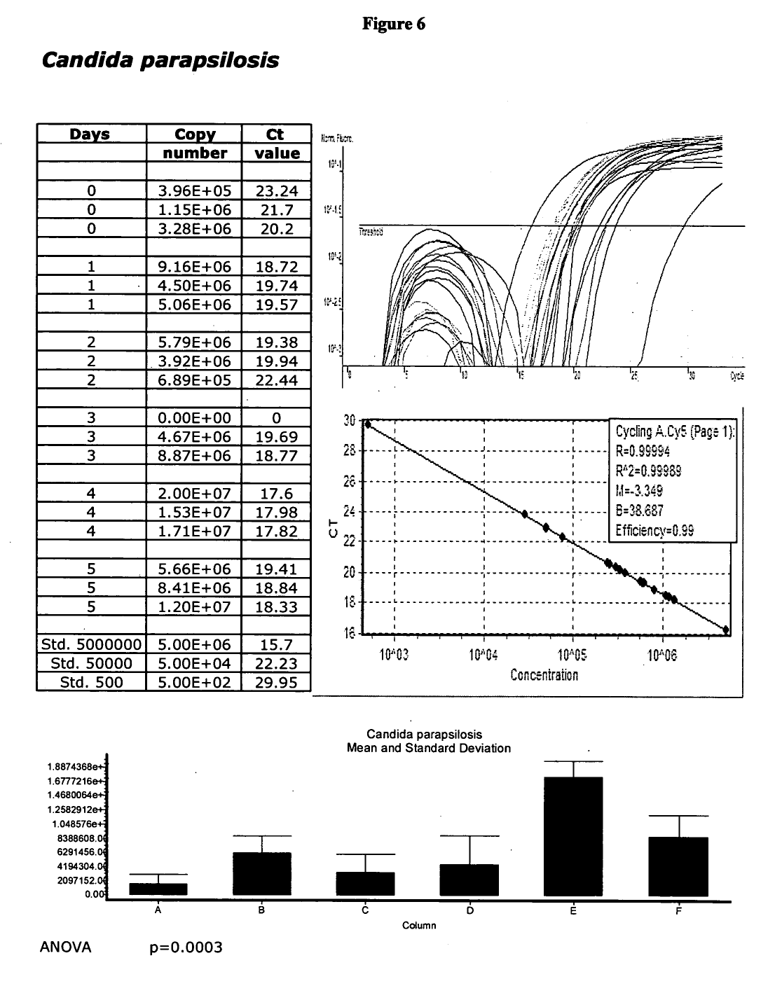

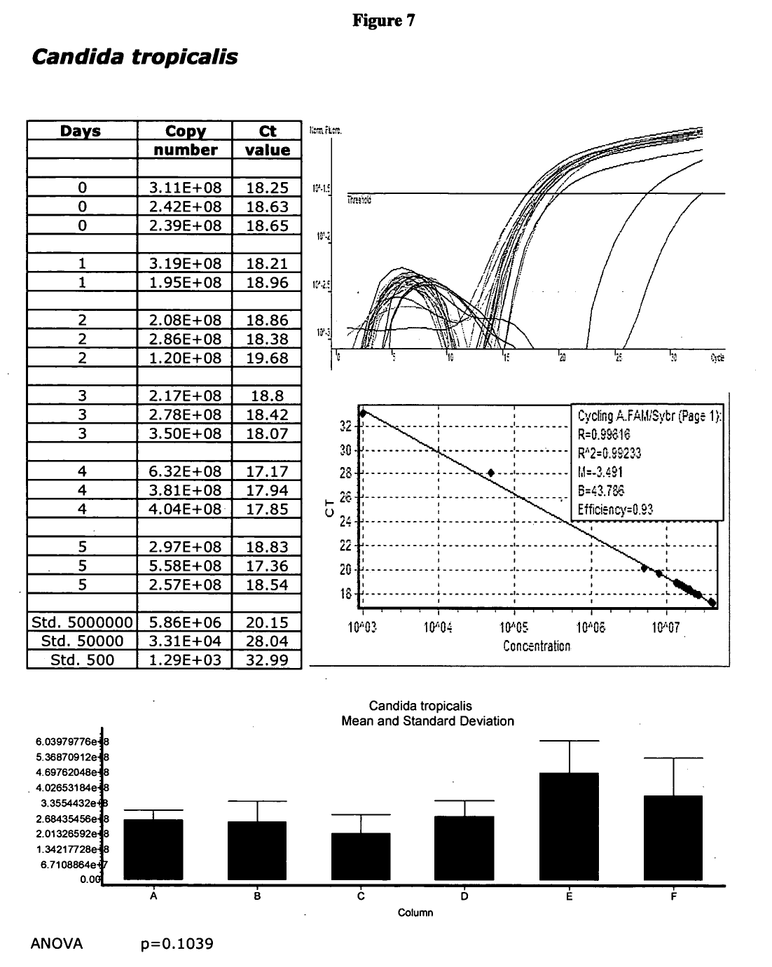

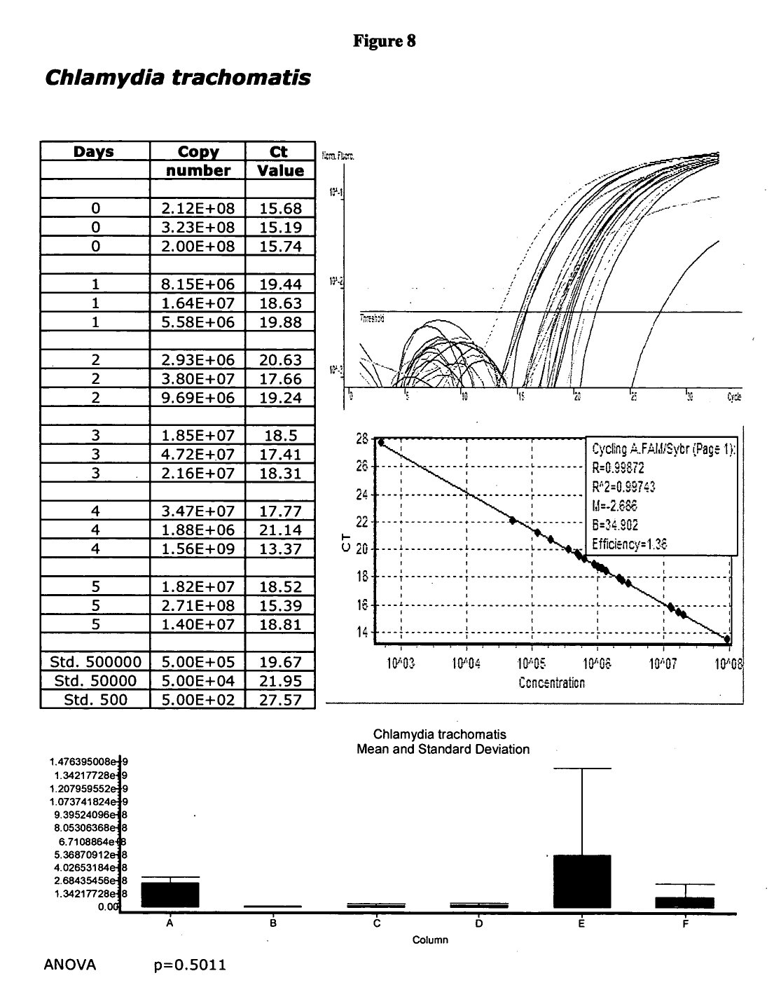

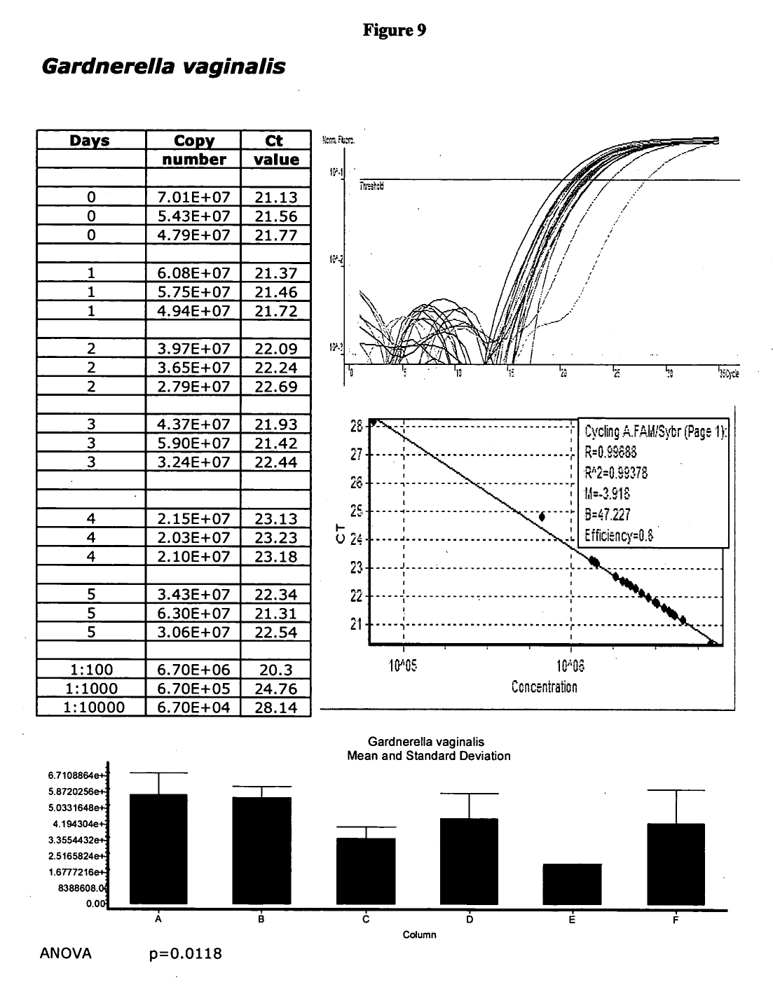

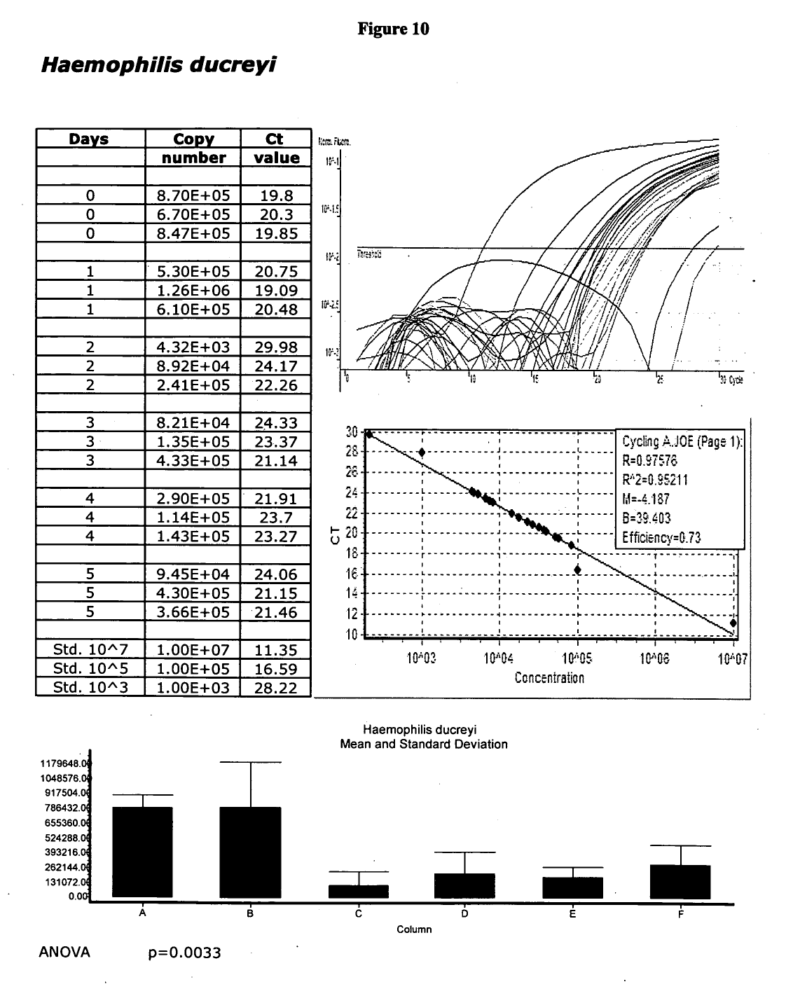

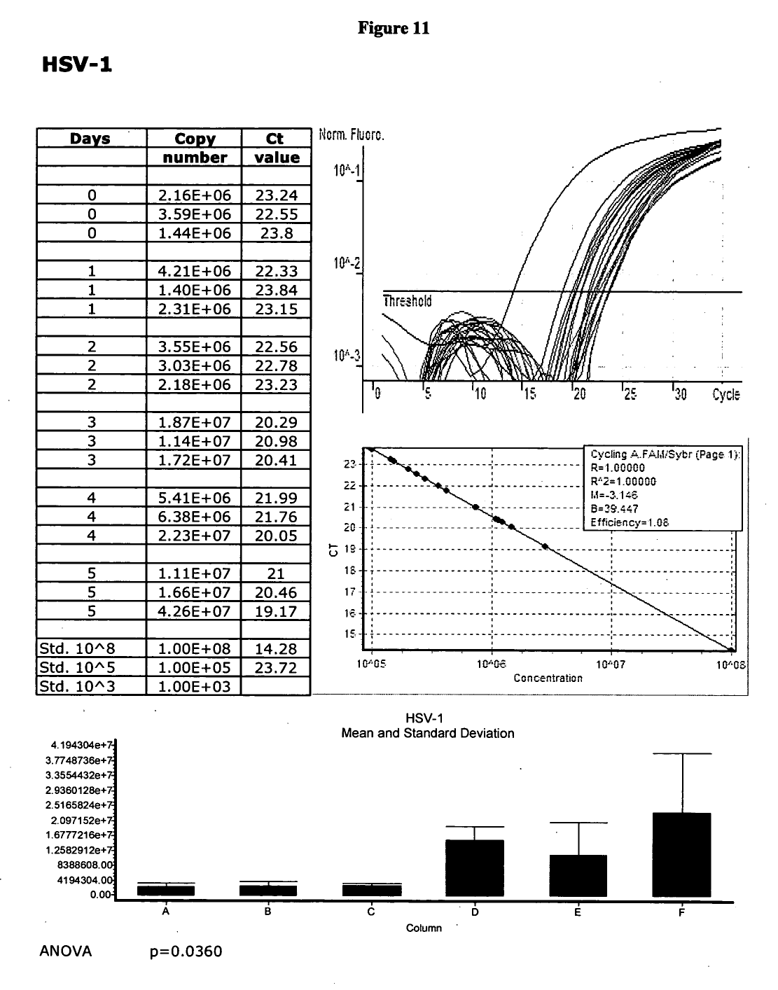

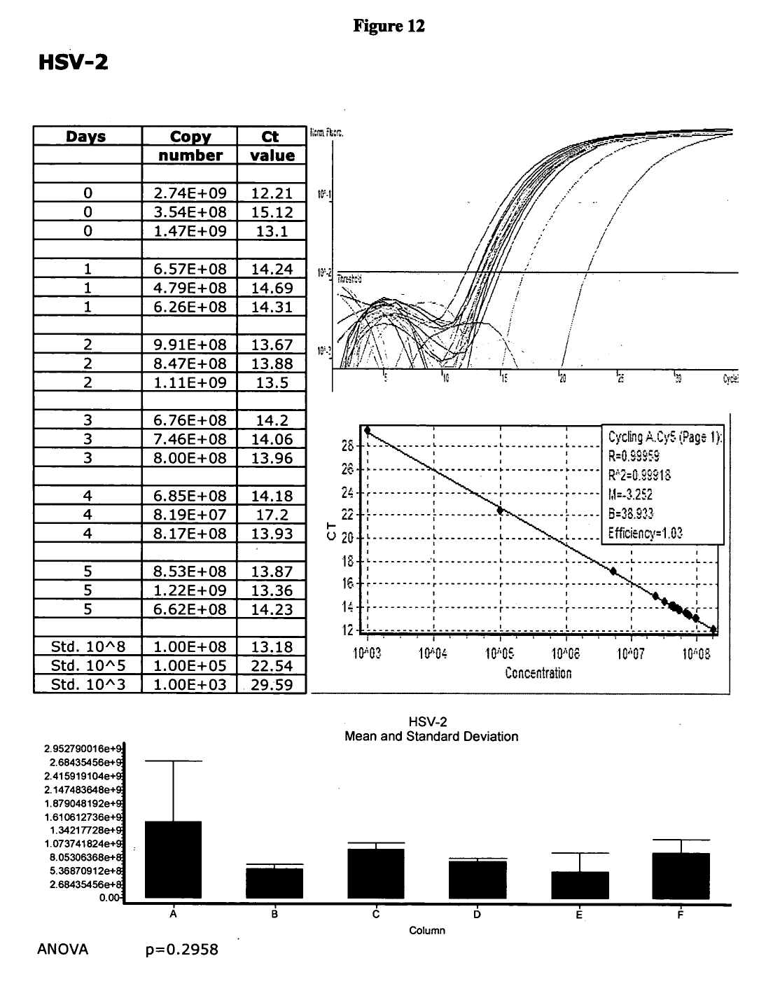

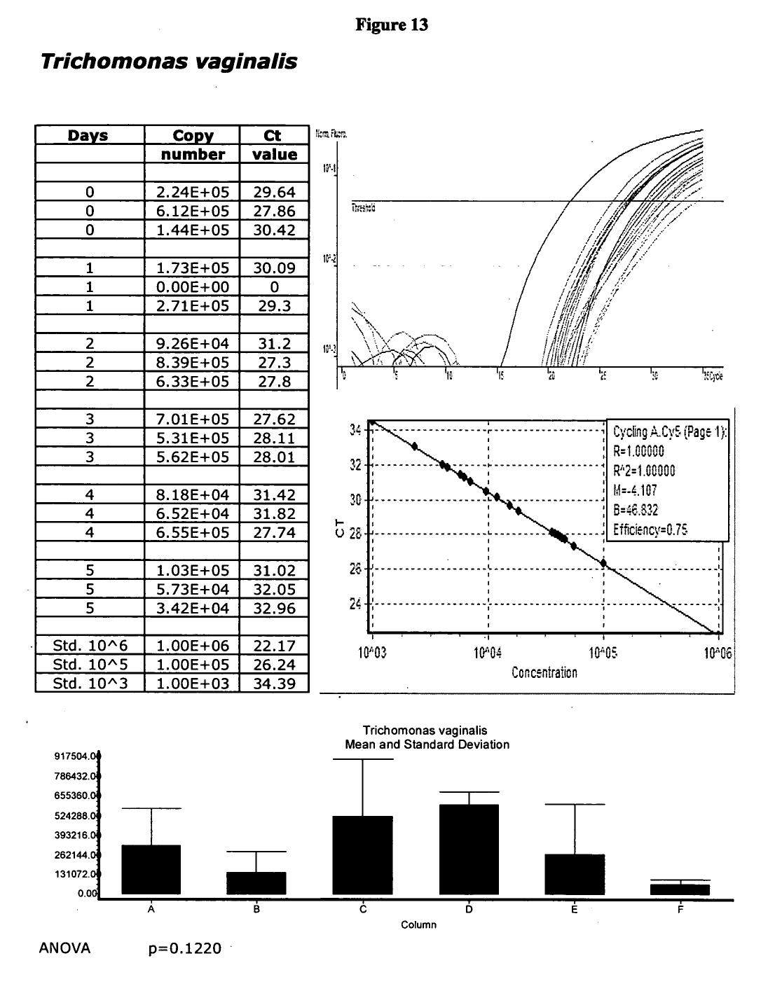

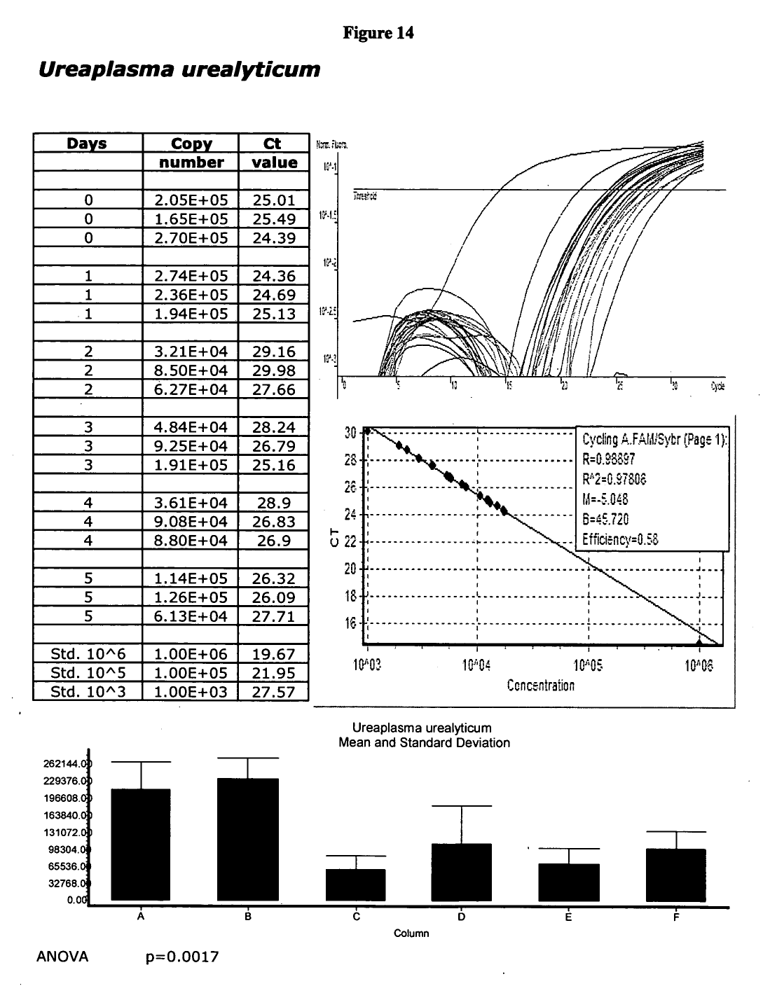

TABLE 5

| | | | # Positive |
|---|---|---|---|
| | | | Time Pts./ |
| | Pathogen | Type of PCR | # Specimens Tested |
| 1 | Bacteriodes fragilis | Conventional PCR | 18/18 (see FIG. 2) |
| 2 | Candida albicans | Real-time PCR | 18/18 (see FIG. 4) |
| 3 | Candida glabrata | Real-time PCR | 14/18 (see FIG. 5) |
| 4 | Candida parapsilosis | Real-time PCR | 18/18 (see FIG. 6) |
| 5 | Candida tropicalis | Real-time PCR | 18/18 (see FIG. 7) |
| 6 | Chlamydia trachomatis | Real-time PCR | 18/18 (see FIG. 8) |
| 7 | Gardnerella vaginalis | Real-time PCR | 18/18 (see FIG. 9) |
| 8 | Haemophilis ducreyi | Real-time PCR | 18/18 (see FIG. 10) |
| 9 | Herpes Simples Virus-1 | Real-time PCR | 18/18 (see FIG. 11) |
| 10 | Herpes Simples Virus-2 | Real-time PCR | 18/18 (see FIG. 12) |
| 11 | Mobiluncus mulieris | Conventional PCR | 18/18 (see FIG. 3) |
| 12 | Mycoplasma hominis | Conventional PCR | 6/6 |
| 13 | Neisseria gonorrhoeae | Real-time PCR | 18/18 |
| 14 | Trichomonas vaginalis | Real-time PCR | 18/18 (see FIG. 13) |
| 15 | Ureaplasma urealyticum | Real-time PCR | 18/18 (see FIG. 14) |

### Example II

### DNA Extraction from Transport Media

For DNA extraction, see, e.g., Goessens et al., 1995, Influence of volume of sample processed on detection of Chlamydia trachomatis in urogenital samples by PCR. Journal of Clinical Microbiology 33:251-253.

The following steps outline the procedure to isolate and purify DNA from transport media. The specimen is submitted as a self-contained unit with transport media.
Proteinase K: 100 μl Tris (pH 7.5), 4.9 ml ddH₂O, 5 ml glycerol. Dissolve well and store at −20° C. as 500 μl aliquots.

10% SDS: 10 g SDS in 100 ml of ddH₂O.

Equipment:

Disposable pipette tips
Disposable transfer pipette
Laboratory timer
1.5 ml microcentrifuge tube
55° C. water bath
Pipettes to deliver a range of 1-1000 μl
   Procedure:
1. Mix the swab thoroughly in the transport media.
2. Pipette 470 μl of transport media into a labeled microcentrifuge tube.
3. Add 25 μl of 10% SDS and 12 μl of Proteinase K. Mix well.
4. Incubate for 2 hours in 55° C. water bath.
5. After 2 hours, place 200 μl of Tris saturated phenol and 200 μl of chloroform:isoamyl alcohol (24:1) in the tube. Shake the tube to mix the layers.
6. Centrifuge at 14,000 rpm for 5 minutes at room temperature. This will separate the layers.
7. Remove the top chloroform layer (containing the DNA) being careful not to pipette any of the bottom or middle layers. Place this into another labeled microcentrifuge tube. The first tube containing the remaining layers may be discarded.
8. To this new tube add 0.1× volume of 3 M sodium acetate. Also add 2× volumes of cold 100% ethanol. Vortex and place in −20° C. overnight.
9. Centrifuge the tube at 14,000 rpm at 4° C. for 10 minutes. This will pellet the precipitated DNA.
10. Remove and discard the supernatant. Add 1000 μl of 70% ethanol to wash the pellet. Slightly resuspend the pellet.
11. Centrifuge the tube again at 14,000 rpm at 4° C. for 5 minutes to form a pellet.
12. Place the tube with the top open into the CentriVap (Labconco, Kansas City, Mo.). Spin at 35° C. for approximately 15 minutes. Spin until the pellet is dry, being very careful not to overdry.
13. Resuspend the pellet in 20 μl of ddH₂O.
14. Quantitate the DNA using a spectrophotometer.

All publications and patents referred to herein are incorporated by reference. Various modifications and variations of the described subject matter will be apparent to those skilled in the art without departing from the scope and spirit of the invention. Although the invention has been described in connection with specific embodiments, it should be understood that the invention as claimed should not be unduly limited to these embodiments. Indeed, various modifications for carrying out the invention are obvious to those skilled in the art and are intended to be within the scope of the following claims.

1. A method for the collection and maintenance of detectability of a plurality of species of microbiological agents selected from the group consisting of bacteria, fungi, and viruses, in a single gynecological sample comprising:
providing transport media in a resealable container and instructions for preparation and handling of a gynecological sample and an indication of the detectability of the plurality of species wherein the plurality of species comprise at least one species selected from the group consisting of Molluscum contagiosum Virus, Mycoplasma genitalium, Mycoplasma hominis, Candida dubliniensis, Candida krusei, Candida lusitaneae, Atopobium vaginae, erythromycin-resistant Streptococcus agalactiae, clindamycin-resistant Streptococcus agalactiae, Lymphogranuloma venereum, HPV-16, HPV-18, HPV-31, HPV-33, HPV-35, HPV-39, HPV-45, HPV-51, HPV-52, HPV-56, HPV-58, HPV-59, HPV-66, HPV-68, HPV-6/11, HPV-42, HPV-43, and HPV-44.

2. The method according to claim 1 wherein a presence of any species within the plurality of species is indicative of at least one gynecological disorder.

3. The method according to claim 2 wherein the presence of any species within the plurality of species is indicative of an infectious disease or vaginosis.

4. The method according to claim 1 wherein the plurality of species is selected from the group consisting of Bacteroides fragilis, Candida albicans, Candida glabrata, Candida parapsilosis, Candida tropicalis, Chlamydia trachomatis, Gardnerella vaginalis, Haemophilis ducreyi, Herpes simplex virus subtype 1 (HSV-1), Herpes simplex virus subtype 2 (HSV-2), Human papillomavirus (HPV), Mobiluncus mulieris, Mobiluncus curtisii, Molluscum contagiosum Virus, Mycoplasma genitalium, Mycoplasma hominis, Neisseria gonorrhoeae, Treponema pallidum, Trichomonas vaginalis, Ureaplasma urealyticum, and Streptococcus agalactiae (Group B Streptococcus).

5. The method according to claim 4 for the collection and maintenance of the detectability of at least 3 species within the plurality of species.

6. The method according to claim 5 for the collection and maintenance of the detectability of at least 4 species within the plurality of species.

7. The method according to claim 6 for the collection and maintenance of the detectability of at least 5 species within the plurality of species.

8. The method according to claim 7 for the collection and maintenance of the detectability of at least 6 species within the plurality of species.

9. The method according to claim 4 for the collection and maintenance of the detectability of the plurality of species comprising Neisseria gonorrhoeae and Chlamydia trachomatis.

10. The method according to claim 9 for the collection and maintenance of the detectability of the plurality of species comprising Trichomonas vaginalis.

11. The method according to claim 4 for the collection and maintenance of the detectability of the plurality of species comprising Gardnerella vaginalis, Mobiluncus mulieris, Mobiluncus curtisii, and Bacteroides fragilis.

12. The method according to claim 4 for the collection and maintenance of the detectability of the plurality of species comprising Candida albicans, Candida glabrata, Candida parapsilosis, and Candida tropicalis.

13. The method according to claim 4 for the collection and maintenance of the detectability of the plurality of species comprising Herpes simplex virus subtype 1 (HSV-1) and Herpes simplex virus subtype 2 (HSV-2).

14. The method according to claim 13 for the collection and maintenance of the detectability of the plurality of species comprising Treponema pallidum and Haemophilis ducreyi.

15. A kit for the collection and maintenance of detectability of a plurality of species of microbiological agents selected from the group consisting of bacteria, fungi, viruses, and protozoa, in a single gynecological sample comprising:
transport media in a resealable container, a sterile swab, and instructions for preparation and handling of a gynecological sample and a writing which indicates the detectability of the plurality of species wherein the plurality of species comprise at least one species selected from the group consisting of Molluscum contagiosum Virus, Mycoplasma genitalium, Mycoplasma hominis, Candida dubliniensis, Candida krusei, Candida lusitaneae, Atopobium vaginae, erythromycin-resistant Streptococcus agalactiae, clindamycin-resistant Streptococcus agalactiae, Lymphogranuloma venereum, HPV-16, HPV-18, HPV-31, HPV-33, HPV-35, HPV-39, HPV-45, HPV-51, HPV-52, HPV-56, HPV-58, HPV-59, HPV-66, HPV-68, HPV-6/11, HPV-42, HPV-43, and HPV-44.

16. The kit according to claim 15 wherein the plurality of species is selected from the group consisting of Bacteroides fragilis, Candida albicans, Candida glabrata, Candida parapsilosis, Candida tropicalis, Chlamydia trachomatis, Gardnerella vaginalis, Haemophilis ducreyi, Herpes simplex virus subtype 1 (HSV1), Herpes simplex virus subtype 2 (HSV2), Human papillomavirus (HPV), Mobiluncus mulieris, Mobiluncus curtisii, Molluscum contagiosum Virus, Mycoplasma genitalium, Mycoplasma hominis, Neisseria gonorrhoeae, Treponema pallidum, Trichomonas vaginalis, Ureaplasma urealyticum, and Streptococcus agalactiae (Group B Streptococcus).

17. The kit according to claim 16 for the collection and maintenance of the detectability of at least 3 species within the plurality of species.

18. The kit according to claim 17 for the collection and maintenance of the detectability of at least 4 species within the plurality of species.

19. A method of using a kit for collection and maintenance of detectability of a plurality of species, wherein the kit comprises transport media in a resealable container and instructions for preparation and handling of a gynecological sample and an indication of the detectability of a plurality of species of microbiological agents selected from the group consisting of bacteria, fungi, viruses, and protozoa in the gynecological sample, comprising:
collecting a clinical sample of a gynecological environment in the transport media wherein the plurality of species comprise at least one species selected from the group consisting of Molluscum contagiosum Virus, Mycoplasma genitalium, Mycoplasma hominis, Candida dubliniensis, Candida krusei, Candida lusitaneae, Atopobium vaginae, erythromycin-resistant Streptococcus agalactiae, clindamycin-resistant Streptococcus agalactiae, Lymphogranuloma venereum, HPV-16, HPV-18, HPV-31, HPV-33, HPV-35, HPV-39, HPV45, HPV-51, HPV-52, HPV-56, HPV-58, HPV-59, HPV-66, HPV-68, HPV-6/11, HPV-42, HPV-43, and HPV-44.

20. The method according to claim 19 wherein the plurality of species is selected from the group consisting of Bacteroides fragilis, Candida albicans, Candida glabrata, Candida parapsilosis, Candida tropicalis, Chlamydia trachomatis, Gardnerella vaginalis, Haemophilis ducreyi, Herpes simplex virus subtype 1 (HSV1), Herpes simplex virus subtype 2 (HSV2), Human papillomavirus (HPV), Mobiluncus mulieris, Mobiluncus curtisii, Molluscum contagiosum Virus, Mycoplasma genitalium, Mycoplasma hominis, Neisseria gonorrhoeae, Treponema pallidum, Trichomonas vaginalis, Ureaplasma urealyticum, and Streptococcus agalactiae (Group B Streptococcus).
